(19) United States
(12) Patent Application Publication
(10) Pub. No.: US 2015/0031555 A1
(43) Pub. Date: Jan. 29, 2015

(54) METHOD FOR CORRECTION OF BIAS IN MULTIPLEXED AMPLIFICATION

(71) Applicant: GIGAGEN, INC., San Francisco, CA (US)

(72) Inventors: David Scott Johnson, San Francisco, CA (US); Andrea Loehr, San Francisco, CA (US)

(21) Appl. No.: 14/374,371

(22) PCT Filed: Jan. 24, 2013
(86) PCT No.: PCT/US2013/022843

§ 371 Date: Jul. 24, 2014

Related U.S. Application Data
(60) Provisional application No. 61/590,087, filed on Jan. 24, 2012.

(51) Int. Cl. C12Q 1/68; G06F 19/24

(52) U.S. Cl. CPC C12Q 1/6851; G06F 19/24; C12Q 1/6881; C12Q 2600/156
USPC 506/2; 506/9; 702/19

(57) ABSTRACT

This invention relates a method to correct for bias inherent to multiplexed sequence amplification. The resulting corrected data is a much more accurate representation of true quantities than unprocessed data.

### CROSS REFERENCE TO RELATED APPLICATIONS

This application claims the benefit of U.S. Provisional Application No. 61/590,087 filed Jan. 24, 2012, which is hereby incorporated by reference in its entirety.

### STATEMENT REGARDING FEDERALLY SPONSORED RESEARCH OR DEVELOPMENT

This invention was made with government support under grant number IIP-1111480 awarded by the National Science Foundation. The United States Government has certain rights in the invention.

### 1. FIELD OF THE INVENTION

This invention relates a method to correct for bias inherent to multiplexed sequence amplification. The resulting corrected data is a much more accurate representation of true quantities than unprocessed data.

### 2. BACKGROUND OF THE INVENTION

### 2.1. Introduction

Immune systems are comprised of a huge diversity of immune cells, such as T cells and B cells Immune cell repertoires are comprised of millions of clones, which produce proteins that enable each cell to specifically recognize a single antigen. When the cells recognize that antigen, they produce an immune response. Genetic analysis of millions of immune cells is useful in medicine and research, in part because components of an individual's immune system are indicative of health. Disregulation of the immune system is responsible for a variety of disorders including autoimmune diseases such as Crohn's disease, juvenile diabetes (Type 1 diabetes, T1D), multiple sclerosis, rheumatoid arthritis, and systemic lupus erythromatosis (SLE) Immune monitoring is useful to better understand cancer, immunotherapy, and immune-competence. In addition, detailed analysis of the immune system can determine appropriate donors for organ transplants and monitor for signs of graft versus host disease (GVHD).

Antibodies are produced by recombined genomic immunoglobulin (Ig) sequences in B lineage cells. Immunoglobulin light chains are derived from either κ or λ genes. The λ genes are comprised of four constant (C) genes and approximately thirty variable (V) genes. In contrast, the κ genes are comprised of one C gene and 250 V genes. The heavy chain gene family is comprised of several hundred V genes, fifteen D genes, and four joining (J) genes. Somatic recombination during B cell differentiation randomly chooses one V-D-J combination in the heavy chain and one V-J combination in either κ or λ light chain. Because there are so many genes, millions of unique combinations are possible. The V genes also undergo somatic hypermutation after recombination, generating further diversity. Despite this underlying complexity, it is possible to use dozens of primers targeting conserved sequences to sequence the full heavy and light chain complement in several multiplexed reactions (van Dongen et al., 2003 Leukemia 17: 2257-2317).

T cells use T cell receptors (TCR) to recognize antigens and control immune responses. The T cell receptor is composed of two subunits: α and β or γ and δ. Much of the peptide variability of the TCR is encoded in complementary determining region 3β (CDR3β), which is formed by recombination between noncontiguous variable (V), diversity (D), and joining (J) genes in the b chain loci (Wang et al., 2010 PNAS 107:1518-23). A published set of forty-five forward primers and thirteen reverse primers amplify the ˜200 bp recombined genomic CDR3β region for multiplex amplification of the full CDR3β complement of a sample of human peripheral blood mononuclear cells (Robins et al., 2009 Blood 114:4099-4107; Robins et al., 2010 Science Translational Med 2:47ra64). The CDR3β region begins with the second conserved cysteine in the 3′ region of the Vβ gene and ends with the conserved phenylalanine encoded by the 5′ region of the Jβ gene (Monod et al., 2004 Bioinformatics 20:i379-i385). Thus, amplified sequences can be informatically translated to locate the conserved cysteine, obtain the intervening peptide sequence, and tabulate counts of each unique clone in the sample.

Several groups have pending or granted patents comprising molecular methods for multiplexed immune repertoire analysis by PCR and deep sequencing. Han (WO 2009/137255) describes a protocol and primer system for amplification of immune repertoires. Lim et al. (WO 2005/059176) also describes a very similar multiplexed method. Fahem & Willis (WO 2010/053587) describes a molecular system and method for multiplexed molecular analysis of immune repertoires that is similar to Han and Lim.

However, these protocols are all prone to amplification bias. Bias can be mitigated chemically through careful optimization of factors such as primer design, annealing temperature, buffer composition, and PCR cycle number. See for example, Markoulatos et al., 2002 J Clin Lab Anal 16: 47-51. Alternatively, bias can be corrected by computational methods. If bias is consistent among experiments, depending on the nature of the underlying sequences, it is possible to correct raw data using models built from prior knowledge of said amplification bias.

This invention uses a predefined control library of known immunological sequences, builds a mathematical model for each sequence, and then uses the mathematical model to correct amplification bias in experimental samples.

### 3. SUMMARY OF THE INVENTION

In particular non-limiting embodiments, the invention is directed to a method for preparing a series of mathematical functions for correction of bias in amplification of a plurality of immune related sequences which comprises: (a) amplifying a first mixture comprising at least two different immune related sequences at known concentrations; (b) amplifying a second mixture comprising the immune related sequences of step (a) wherein the sequences are present at different concentrations than the first mixture; (c) measuring sequence counts for the first and second amplified mixtures of immune related sequences; (d) generating a plurality of mathematical functions for correction of bias that model relationships between concentrations and measured sequence counts; (e) assembling the mathematical functions of step (d) to generate the series of mathematical functions useful to correct amplification bias.

The invention is also directed to a computer-implemented method for correcting bias in a plurality of immune related sequences which comprises: (a) obtaining a plurality of measurements of levels of amplified immune related sequences from an unknown sample; and (b) using an assembled series of mathematical functions and the measurements from step (a) to correct amplification bias in amplified immune related sequences from the unknown sample.

In addition, the invention is directed to a system for correcting bias in amplified immune related sequences which comprises: (a) an assembler module comprising an assembled series of mathematical functions useful to correct amplification bias in a plurality of immune related sequences; and (b) a calculating module that corrects bias in amplification of immune related sequences using the assembled series of mathematical functions and measurements of levels of amplified immune related sequences from an unknown sample.

### 4. BRIEF DESCRIPTION OF THE FIGURES

FIG. 1. Schematic overview of how the invention uses informatics to correct raw molecular data for the TCRβ embodiment.

FIG. 2. Conceptual plot of how the invention uses informatics to correct raw molecular data. In this embodiment, measurements are made for one V-J pair at five different concentrations (gray circles). A linear model is fit to the measurements (dotted line). If the measurements were unbiased (black circles), the assumption is that the data follow y=x (solid line). Such an assumption would lead to false conclusions from the empirical data. The linear model can later be used to correct bias informatically.

FIG. 3. Plot of how the invention uses informatics to correct raw molecular data. In this embodiment, measurements are made for one V-J pair at four different concentrations (circles). A linear model is fit to the measurements (solid line). If the measurements are unbiased, the assumption is that the data follow y(x)=x (dashed line). Such an assumption would lead to false conclusions from the empirical data. The linear model is used to correct bias informatically, reconciling the biased measurements (circles) with the unbiased case (dashed line) as demonstrated by the corrected data (solid squares).

### 5. DETAILED DESCRIPTION OF THE INVENTION

### 5.1. Definitions

Terms used in the claims and specification are defined as set forth below unless otherwise specified.

The term “B cell” refers to a type of lymphocyte that plays a large role in the humoral immune response (as opposed to the cell-mediated immune response, which is governed by T cells). The principal functions of B cells are to make antibodies against antigens, perform the role of antigen-presenting cells (APCs) and eventually develop into memory B cells after activation by antigen interaction. B cells are an essential component of the adaptive immune system.

The term “bulk sequencing” or “next generation sequencing” or “massively parallel sequencing” refers to any high throughput sequencing technology that parallelizes the DNA sequencing process. For example, bulk sequencing methods are typically capable of producing more than one million polynucleic acid amplicons in a single assay. The terms “bulk sequencing,” “massively parallel sequencing,” and “next generation sequencing” refer only to general methods, not necessarily to the acquisition of greater than 1 million sequence sequences in a single run. Any bulk sequencing method can be implemented in the invention, such as reversible terminator chemistry (e.g., Illumina), pyrosequencing using polony emulsion droplets (e.g., Roche), ion semiconductor sequencing (IonTorrent), single molecule sequencing (e.g., Pacific Biosciences), massively parallel signature sequencing, etc.

The term “cell” refers to a functional basic unit of living organisms. A cell includes any kind of cell (prokaryotic or eukaryotic) from a living organism. Examples include, but are not limited to, mammalian mononuclear blood cells, yeast cells, or bacterial cells.

The term “ligase chain reaction” or LCR refers to a type of DNA amplification where two DNA probes are ligated by a DNA ligase, and a DNA polymerase is used to amplify the resulting ligation product. Traditional PCR methods are used to amplify the ligated DNA sequence.

The term “mammal” as used herein includes both humans and non-humans and include, but is not limited to, humans, non-human primates, canines, felines, murines, bovines, equines, and porcines.

The term “polymerase chain reaction” or PCR refers to a molecular biology technique for amplifying a DNA sequence from a single copy to several orders of magnitude (thousands to millions of copies). PCR relies on thermal cycling, which requires cycles of repeated heating and cooling of the reaction for DNA melting and enzymatic replication of the DNA. Primers (short DNA fragments, or oligonucleotides) containing sequences complementary to the target region of the DNA sequence and a DNA polymerase are key components to enable selective and repeated amplification. As PCR progresses, the DNA generated is itself used as a template for replication, setting in motion a chain reaction in which the DNA template is exponentially amplified. A heat-stable DNA polymerase, such as Taq polymerase, is used. The thermal cycling steps are necessary first to physically separate the two strands in a DNA double helix at a high temperature in a process called DNA melting. At a lower temperature, each strand is then used as the template in DNA synthesis by the DNA polymerase to selectively amplify the target DNA. The selectivity of PCR results from the use of primers that are complementary to the DNA region targeted for amplification under specific thermal cycling conditions.

The term “reverse transcriptase polymerase chain reaction” or RT-PCR refers to a type of PCR reaction used to generate multiple copies of a DNA sequence. In RT-PCR, an RNA strand is first reverse transcribed into its DNA complement (complementary DNA or cDNA) using the enzyme reverse transcriptase, and the resulting cDNA is amplified using traditional PCR techniques.

The term “T cell” refers to a type of cell that plays a central role in cell-mediated immune response. T cells belong to a group of white blood cells known as lymphocytes and can be distinguished from other lymphocytes, such as B cells and natural killer T (NKT) cells by the presence of a T cell receptor (TCR) on the cell surface. T cells responses are antigen specific and are activated by foreign antigens. T cells are activated to proliferate and differentiate into effector cells when the foreign antigen is displayed on the surface of the antigen-presenting cells in peripheral lymphoid organs. T cells recognize fragments of protein antigens that have been partly degraded inside the antigen-presenting cell. There are two main classes of T cells—cytotoxic T cells and helper T cells. Effector cytotoxic T cells directly kill cells that are infected with a virus or some other intracellular pathogen. Effector helper T cells help to stimulate the responses of other cells, mainly macrophages, B cells and cytotoxic T cells.

The term “gene” refers to a nucleic acid sequence that can be potentially transcribed and/or translated which may include the regulatory elements in 5′ and 3′, and the introns, if present. Examples of genes are TRBV10-6, TRBJ2-7. See “gene” at www.imgt.org.

The term “group” a set of genes which share the same gene type and participate potentially to the synthesis of a polypeptide of the same immunologic chain type. By extension, a group includes the related pseudogenes and orphans. A group is independent from the species. Groups are defined for the immunoglobulins (IG), T cell receptors (TR) and major histocompatibility complex (MHC) molecules, e.g., TRBJ, TRBV and TRBD are part of the same group. See “group” at www.imgt.org.

The term “subgroup” refers to a set of IG or TR genes (C-gene, V-gene, D-gene or J-gene) which belong to the same group, in a given species, and which share at least 75% identity at the nucleotide level (in the germline configuration for V, D, and J), e.g., TRBV6-1 and TRBV6-2 are genes in the TRBV6 subgroup. See “subgroup” in www.imgt.org.

It must be noted that, as used in the specification and the appended claims, the singular forms “a,” “an,” and “the” include plural referents unless the context clearly dictates otherwise.

### 5.2. General Methods

In embodiment 1, the invention is directed to a method for preparing a series of mathematical functions for correction of bias in amplification of a plurality of immune related sequences which comprises: (a) amplifying a first mixture comprising at least two different immune related sequences at known concentrations; (b) amplifying a second mixture comprising the immune related sequences of step (a) wherein the sequences are present at different concentrations than the first mixture; (c) measuring sequence counts for the first and second amplified mixtures of immune related sequences; (d) generating a plurality of mathematical functions for correction of bias that model relationships between concentrations and measured sequence counts; (e) assembling the mathematical functions of step (d) to generate the series of mathematical functions useful to correct amplification bias.

In embodiment 1, the immune related sequences may be subcloned into a circular vector; multiplexed polymerase chain reaction may be used to amplify the immune related sequences.

In embodiment 1, the immune related sequences are immunoglobulin IgH or immunoglobulin IgL sequences; T cell receptor sequences; joining (J) gene sequences; or variable (V) gene sequences.

In embodiment 1, greater than forty immune related sequences may be selected from possible combinations of joining (J) and variable (V) gene sequences. Alternatively, more than six different immune related sequences are used in the first mixture and the concentration differences for at least one immune related sequence is greater than three orders of magnitude in the second mixture.

In embodiment 1, the mathematical function may be a linear or nonlinear equation.

The invention is also directed to embodiment 2, a method for correction of bias in amplification of in an immune repertoire sample, comprising: (a) amplifying of an immune repertoire sample; (b) obtaining at least 1,000 sequences from the amplified immune repertoire sample; and (c) correcting levels generated from amplification at least one sequence in the immune repertoire sample using at least one mathematical function from the series of mathematical functions generated in embodiment 1.

In embodiment 2, massively parallel sequencing may be used to generate sequences from the immune repertoire sample; at least 10,000 sequences are obtained from the immune repertoire sample; or at least 100,000 sequences are obtained from the immune repertoire sample.

The invention is also directed to embodiment 3, a computer-implemented method for correcting bias in a plurality of immune related sequences which comprises: (a) obtaining a plurality of measurements of levels of amplified immune related sequences from an unknown sample; and (b) using an assembled series of mathematical functions and the measurements from step (a) to correct amplification bias in amplified immune related sequences from the unknown sample. In the computer-implemented method for correcting bias, step (a) and step (b) may be carried out automatically.

In addition, the invention is directed to a system for correcting bias in amplified immune related sequences which comprises: (a) an assembler module comprising an assembled series of mathematical functions useful to correct amplification bias in a plurality of immune related sequences; and (b) a calculating module that corrects bias in amplification of immune related sequences using the assembled series of mathematical functions and measurements of levels of amplified immune related sequences from an unknown sample.

The methods of the invention described herein may be applied to correct a variety of sources of bias in amplification using PCR including, but not limited to, PCR selection bias and PCR drift. Wagner et al., 1994, Syst Biol 43(2) 250-261.

### 5.3. Use of the Methods

Methods of the invention are applied to post-transplant immune monitoring whether autologous, allogeneic, syngeneic, or xenographic. After an allogeneic transplant (i.e., kidney, liver, or stem cells), a host's T cells response to transplants are assessed to monitor the health of the host and the graft. Molecular monitoring of blood or urine is helpful to detect acute or chronic rejection before a biopsy would typically be indicated. For example, detection of alloantibodies to human leukocyte antigen (HLA) has been associated with chronic allograft rejection (Terasaki and Ozawa, 2004 American Journal of Transplantation 4:438-43). Other molecular markers include b2-microglobulin, neopterin, and proinflammatory cytokines in urine and blood (Sabek et al., 2002 Transplantation 74:701-7; Tatapudi et al., 2004 Kidney International 65:2390; Matz et al., 2006 Kidney International 69:1683; Bestard et al., 2010 Current Opinion in Organ Transplantation 15:467-473). However, none of these methods has become widely adopted in clinical practice, perhaps due to low specificity and sensitivity. Prior work has shown that regulatory T cells (Treg) induce graft tolerance by down-regulating helper T cells (Th) (Graca et al., 2002 Journal of Experimental Medicine 195: 1641). Additionally, transplanting hematopoietic stem cells from HLA-mismatched donors into the recipient has resulted in long-term nonimmunosuppressive renal transplant tolerance up to 5 years after transplant (Kawai et al., 2008 NEJM 358:353-61).

### 5.4. T Cell Analysis and Latent Tuberculosis Diagnosis

Latent tuberculosis (TB) is a major global epidemic, affecting as many as 2 billion people worldwide. There is currently no reliable test for clinical diagnosis of latent TB. This technology gap has severe clinical consequences, since reactivated TB is the only reliable hallmark of latent TB. Furthermore, clinical trials for vaccines and therapies lack biomarkers for latent TB, and therefore must follow cohorts over many years to prove efficacy.

The major current vaccine for tuberculosis, bacillus Calmette-Guérin (BCG), is an unreliable prophylactic. In a meta-analysis of dozens of epidemiological studies, the overall effect of BCG was 50% against TB infections, 78% against pulmonary TB, 64% against TB meningitis, and 71% against death due to TB infection (Colditz et al., 1994 JAMA 271:698-702). Additionally, the rapid rise in multidrug resistant TB has increased the need for new vaccine and immunotherapy approaches. Up to 90% of infected, immunocompetent individuals never progress to disease, resulting in the huge global latent TB reservoir (Kaufmann, 2005 Trends in Immunology 26:660-67).

Since tuberculosis is a facultative intracellular pathogen, immunity is almost entirely mediated through T cells. Interferon-g expressing T helper 1 (Th1) cells elicit primary TB response, with some involvement by T helper 2 cells (Th2). After primary response, the bacteria become latent, controlled by regulatory T cell (Treg) and memory T cells (Tmem). Recently, eleven new vaccine candidates have entered clinical trials (Kaufmann, 2005 Trends in Immunology 26:660-67). These vaccines are all “post-exposure” vaccines, i.e., they target T cell responses to latent TB and are intended to prevent disease reactivation. Because of the partial failure of BCG to induce full immunity, rational design and validation of future TB vaccines should include systematic analysis of the specific immune response to both TB and the new vaccines.

For decades, the standard of care for diagnosis of latent tuberculosis has been the tuberculin skin test (TST) (Pai et al., 2004 Lancet Infectious Disease 4:761-76). More recently, two commercial in vitro interferon-g assays have been developed: the QuantiFERON-TB assay and the T SPOT-TB assay. These assays measure cell-mediated immunity by quantifying interferon-g released from T cells when challenged with a cocktail of tuberculosis antigens. Unfortunately, neither the TST nor the newer interferon-g tests is effective at distinguishing latent TB from cleared TB (Diel et al., 2007 American Journal of Respir Crit Care Med 177:1164-70). This is a significant problem because patients without clinical evidence of latent TB (i.e., visualization of granulomas) but with positive TST or interferon-g test typically receive 6-9 months of isoniazide therapy, even though this empiric intervention is unnecessary in patients who have cleared primary infection and can cause serious complications such as liver failure.

Prior work has demonstrated that T cell responses are used to distinguish latent from active TB (Schuck et al., 2009 PLoS One 4:e5590). The premise of this prior work is that immune cells directed against TB antigens will be expanded in the memory T cell population if the TB is latent, but expanded in a helper T cell fraction if the TB is active. Functional T cell sequencing is used to distinguish latent TB from cleared TB.

### 5.5. T Cell Analysis and Diagnosing or Monitoring Disease

Similarly, functional T cell monitoring is used for diagnosis and monitoring of nearly any human disease. These diseases, include but are not limited to, systemic lupus erythmatosis (SLE), allergy, autoimmune disease, heart transplants, liver transplants, bone marrow transplants, lung transplants, solid tumors, liquid tumors, myelodysplastic syndrome (MDS), chronic infection, acute infection, hepatitis, human papilloma virus (HPV), herpes simplex virus, cytomegalovirus (CMV), and human immunodeficiency virus (HIV). Such monitoring includes individual diagnosis and monitoring or population monitoring for epidemiological studies.

T cell monitoring is used for research purposes using any non-human model system, such as zebrafish, mouse, rat, or rabbit. T cell monitoring also is used for research purposes using any human model system, such as primary T cell lines or immortal T cell lines.

### 5.6. B Cell Analysis and Drug Discovery

Antibody therapeutics are increasingly used by pharmaceutical companies to treat intractable diseases such as cancer (Carter 2006 Nature Reviews Immunology 6:343-357). However, the process of antibody drug discovery is expensive and tedious, requiring the identification of an antigen, and then the isolation and production of monoclonal antibodies with activity against the antigen. Individuals that have been exposed to disease produce antibodies against antigens associated with that disease. Thus, it is possible mine patient immune repertoires for specific antibodies that could be used for pharmaceutical development.

### 5.7. B Cell Analysis and Monitoring Immunity

Humoral memory B cells (Bmem) help mammalian immune systems retain certain kinds of immunity. After exposure to an antigen and expansion of antibody-producing cells, Bmem cells survive for many years and contribute to the secondary immune response upon re-introduction of an antigen. Such immunity is typically measured in a cellular or antibody-based in vitro assay. In some cases, it is beneficial to detect immunity by amplifying, linking, and detecting IgH and light chain immunoglobulin variable regions in single B cells. Such a method is more specific and sensitive than current methods. Massively parallel B cell repertoire sequencing is used to screen for Bmem cells that contain a certain heavy and light chain pairing which is indicative of immunity.

### 5.8. B Cell Analysis and Diagnosing and Monitoring Disease

B cell monitoring is used for diagnosis and monitoring of nearly any human disease. These diseases include, but are not limited to, systemic lupus erythmatosis (SLE), allergy, autoimmune disease, heart transplants, liver transplants, bone marrow transplants, lung transplants, solid tumors, liquid tumors, myelodysplastic syndrome (MDS), chronic infection, acute infection, hepatitis, human papilloma virus (HPV), herpes simplex virus (HSV), cytomegalovirus (CMV), and human immunodeficiency virus (HIV). Such monitoring could include individual diagnosis and monitoring or population monitoring for epidemiologic al studies.

B cell monitoring is also used for research purposes using any non-human model system, such as zebrafish, mouse, rat, or rabbit. B cell monitoring is used for research purposes using any human model system, such as primary B cell lines or immortal B cell lines.

The article “a” and “an” are used herein to refer to one or more than one (i.e., to at least one) of the grammatical object(s) of the article. By way of example, “an element” means one or more elements.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably “comprise”, “consist of”, or “consist essentially of”, the steps, elements, and/or reagents described in the claims.

The following Examples further illustrate the invention and are not intended to limit the scope of the invention.

### 6. EXAMPLES

### 6.1. Protocol Optimization Using 48-Plex Pool of TCR Plasmid Clones

The true content of any particular TCR repertoire is not known, so an endogenous TCR repertoire cannot serve as a gold standard for protocol optimization. A 48-plex pool of mouse TCRβ plasmid clones was designed to act as template for protocol optimization. First, multiplexed amplification was performed of the mouse TCRβ repertoire as described in Example 2 of PCT/US11/65600 filed Dec. 16, 2011. The PCR products were subcloned using the TOPO-TA vector (Life Technologies), transformed post ligation into TOP10 competent cells (Life Technologies), and 48 transformed colonies were picked. Next, the clones were sequenced by Sanger sequencing to identify the TCRβ clonotype sequences. All of the clones were unique, and represented a broad range of possible V-Jβ combinations. The plasmids were then mixed in a single tube, across three orders of magnitude and with six replicates at each concentration.

The 48-plex mixture was used to optimize the TCRβ amplification protocol. The purification methodology after the first and second PCR steps, the number of cycles in the first PCR, and the annealing temperature in the first PCR were optimized. WA PCR column or gel excision for the purification technology were used. Due to spurious mispriming, the first round of PCR produced multiple bands in addition to a major band in the target size range of 150-200 bp. Gel excision removed the undesired material, but the process was tedious and results in loss of up to 75% of the desired material. Protocols with fewer first PCR amplification cycles typically produce less severe amplification bias, whereas amplification bias is typically skewed in protocols with >30 cycles. Annealing temperature controls the stringency of priming events, with lower temperatures producing higher yields but less specificity.

68 Illumina libraries were constructed using the mixture of 48 plasmids and varying protocol parameters as described above. The libraries were sequenced on a next generation sequencing machine (Illumina) to obtain >500 k paired-end 80 bp sequence tags for each library. To analyze the sequencing data, each 2x80 bp sequence tag was aligned to the sequences of the 48 known clonotypes to obtain the best match. The number of tags aligned to each plasmid for each library was counted, and then these results were correlated with the expected ratios of the input plasmid clones. A linear regression analysis to fit each data set was performed (see Table 1: yielding correlation, R² of 1, and a slope of 1. The protocol used 15 cycles of amplification for the first PCR, an annealing temperature of 61° C., PCR column purification after the first PCR, and gel purification following the second PCR.

TABLE 1
Analysis of selected pilot protocol optimization experiments.
R² and slope were computed from a regression analysis
between the observed count of sequences in each library
versus the known input count. Conditions in row 3 (bold)
are an example of an optimized protocol.

| 1st PCR | 1st PCR | 1st PCR | 2nd PCR | | |
|---|---|---|---|---|---|
| Cycles | Ta | Cleanup | Cleanup | R² | Slope |
| 15 | 57 | column | gel | 0.56 | 0.54 |
| 15 | 59 | column | gel | 0.7 | 0.68 |
| 15 | 61 | column | gel | 0.72 | 0.71 |
| 15 | 63 | column | gel | 0.69 | 0.7 |
| 25 | 57 | column | gel | 0.47 | 0.43 |
| 25 | 59 | column | gel | 0.44 | 0.4 |
| 25 | 61 | column | gel | 0.45 | 0.45 |
| 25 | 63 | column | gel | 0.41 | 0.39 |
| 35 | 57 | column | gel | 0.47 | 0.41 |
| 35 | 59 | column | gel | 0.43 | 0.37 |
| 35 | 61 | column | gel | 0.42 | 0.4 |
| 35 | 63 | column | gel | 0.41 | 0.4 |

### 6.2. Constructing a Control Library of TCRβ Clones and Optimizing PCR Conditions Using the Control Library

Additional experiments are performed to build a library of 960 TCRβ clones that contain at least one representative from each of the 650 possible human V-Jβ combinations. This set of clones is used for molecular and statistical optimizations. A plasmid library of human TCRβ is generated as described above in Section 6.1 above. About 3,000 transformant colonies are picked and the clones are sequenced using standard capillary sequencing (e.g., Sequetech). The V-Jβ pairing corresponding to each sequenced clone is identified as described above in Section 6.1. The goal is to obtain at least one representative clone for each V-Jβ pair. If sequencing finds that some V-Jβ pairs are missing, those pairs are rescued by making libraries of TCRβ using only primers for those missing V-Jβ pairs, subcloning, and sequencing. After several rounds, clones are identified for every possible V-Jβ pair. These plasmids are mixed into a single template mixture, with 96 clones at each concentration and 10 different concentrations across three orders of magnitude.

### 6.3. Optimizing PCR Conditions Using the Control Library

Previous experiments have shown that the first PCR amplification causes most of the amplification bias. Additional experiments are performed using the 960-clone pool and next-generation sequencing to further optimize first PCR cycle number. About 60 TCRβ libraries are generated from the plasmid mixture, with four replicates for each of the 15 cycle numbers between 10 and 25. The library mixtures are quantified and ˜4 million sequences are obtained from each library a GAIIx next-gen sequencer (Illumina) The V-Jβ pairing corresponding to each sequenced clone as described above in Section 6.1, and the counts of sequence tags are tallied for each clone in each data set. Prior work has shown that GC content can affect amplification efficiency (Markoulatos et al., 2002). The immense variety of V(D)Jβ combinations result in an assortment GC contents and lengths. The amplification bias is tested after addition of various reagents, such as betaine or magnesium chloride. Approximately 60 TCRβ libraries are generated from the plasmid mixture, with four replicates for each of 15 different buffers. The library mixtures are quantified and ˜4 million sequences are obtained from each library using a GAIIx next-gen sequencer (Illumina) The V-Jβ pairing is identified corresponding to each sequenced clone as described above in Section 6.1, and the counts of sequence tags are tabulated for each clone in each data set.

### 6.4 Correction of PCR Bias Using Statistical Models

One embodiment of the invention is a method for solving the problem of amplification bias in TCRβ multiplex amplification. Specifically, a statistical model is built for the complete TCRβ repertoire, though in other embodiments one can build a statistical model for portions of the TCRβ repertoire.

First, using the methods in Section 6.2, one builds a plasmid library with at least one representative from possible V-Jβ combinations. To build a high-confidence statistical model, we estimate that we will require measurements for each clone at each of ten concentrations. Therefore, we divide the clone library into ten sets of 96 clones each. Then, one makes ten mixtures of all 960 clones, such that each mixture contains 10 sets of 96 clones, each set at a particular concentration across three orders of magnitude. In this way, each set of 96 clones is present at one of the ten concentrations in one of the mixtures. Using an optimized PCR protocol, one then synthesizes 10 replicate sequencing libraries using these 10 clone mixtures, for a total of 100 libraries. The libraries are then tagged with multiplexing barcodes, and pooled into mixtures of 6-10 libraries. We then obtain 4 million sequences from each library using our GAIIx sequencer (Illumina) Finally, we identify the clone corresponding to each next-gen sequence tag, and then tabulate the counts of sequence tags for each clone in each data set.

Next, the empirical sequence data for the 10 sets of 96 clones is used to build a model that corrects for systemic sequence bias. The model adjusts for amplification bias for each possible V-Jβ combination, similar to prior methods used for methylation analysis by PCR (Moskalev et al., 2011, Nucleic Acids Research 39(11) e77 doi:10.1093). A common method to quantitatively study DNA methylation is parallel analysis of sequences either of unreacted or reacted with sodium bisulfite prior to amplification. Bisulfite converts unmethylated cytosine to uracil which is converted to thymine in the PCR amplification. Due to sequence differences after bisulfite reaction, the “DNA may adopt distinct secondary structures or exhibit different melting behavior, which leads to amplification bias.” Moskalev et al. at page 3. To correct for the methylation bias, Moskalev et al. ran at least three separate PCR reactions on calibration sequences having controlled percentage methylation and curve fit using hyperbolic and polynomial regressions. This problem is substantially simpler than the problem of diverse immune repertoires, because immune repertoire analysis involves many multiplexed primers as well as many multiplexed targets. Therefore, single-plex PCR such as those run by Moskalev et al. is a simpler computational problem. Analysis of full immune repertoires requires a novel, inventive approach.

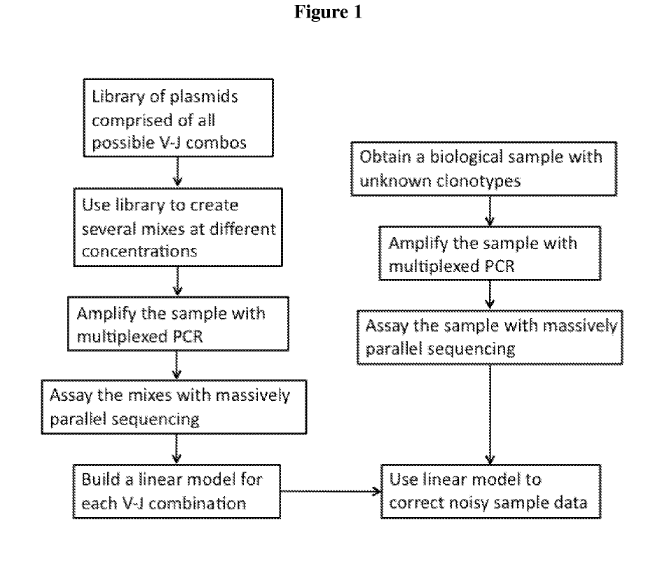

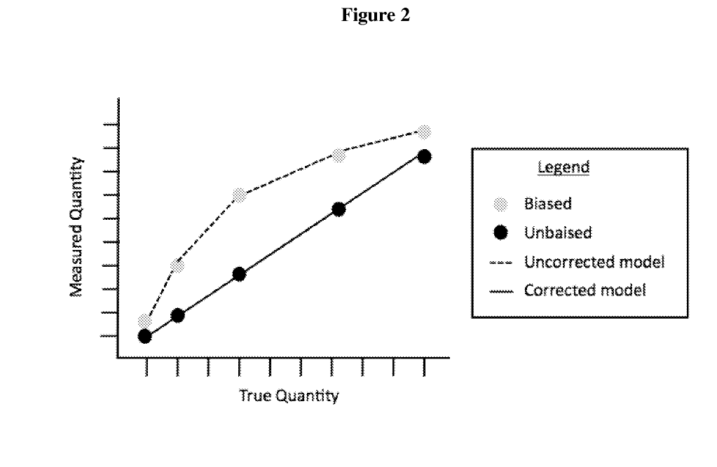

In applicants claimed method, one regularizes sequencing data from each of the 10 sets of 96 clones, such that each clone is expressed as a fraction of the total clone content of the library where is the regularized value for the i^{th} clone, is the empirical count of next-gen sequence tags corresponding to the i^{th} clone, and is the empirical count of next-gen sequence tags corresponding to the j^{th} clone for j=1 . . . 960. Regularization helps prevent over-fitting in the model for each clone. For each of the 960 input clones and using regularized empirical next-gen sequence tag data from the 100 libraries, one next finds the best fit for the function

$$y ( x ) = \frac{{f}_{ma x} mx}{mx - x + {f}_{ma x}}$$

where y is the observed regularized frequency, x is the known input concentration of the clone, m is the slope of the fitted line, and fₘₐₓ is the maximum possible regularized frequency. The slope m reflects the efficiency of primer binding and PCR amplification of a particular clone. The best fit can be calculated using a least-squares method as is routine using open-source scripts in Python. Once one has computed the slope m for a particular clone, one solves the equation y(x) for x and calculates the corrected estimate of the actual number of clones given the empirical count cᵢ. FIG. 1 shows a schematic for both the preparation of the model and its use to correct bias from a sample. FIG. 2 shows a conceptual plot of how the invention uses informatics to correct raw molecular data. In this embodiment, measurements are made for one V-J pair at five different concentrations (gray circles). A linear model is fit to the measurements (dotted line). If the measurements were unbiased (black circles), the assumption is that the data follow y=x (solid line). Such an assumption would lead to false conclusions from the empirical data. The linear model can later be used to correct bias informatically.

To demonstrate the validity of this algorithm given a particular clone set, one skilled in the art can make several new clone mixtures that contain at least 100 new clonotypes at a variety of predefined concentrations across three orders of magnitude. These clone mixtures can then be used to test the algorithm's success at correcting amplification bias.

Such methodology is used for analysis of other kinds of immune repertoires, such as IgH or TCRα. One might use fewer or more clonotypes for building the mathematical model, depending on the clonotypes of interest. Additionally, in certain embodiments one may build models not only for a particular V-J pairing, but for bias correction of one or many genes, groups, or subgroup. For example, in one embodiment, one builds a single model for the full TRBV6 subgroup independent of J unit pairing. Thus, the model corrects for bias in any V-J pairing that contains TRBV6.

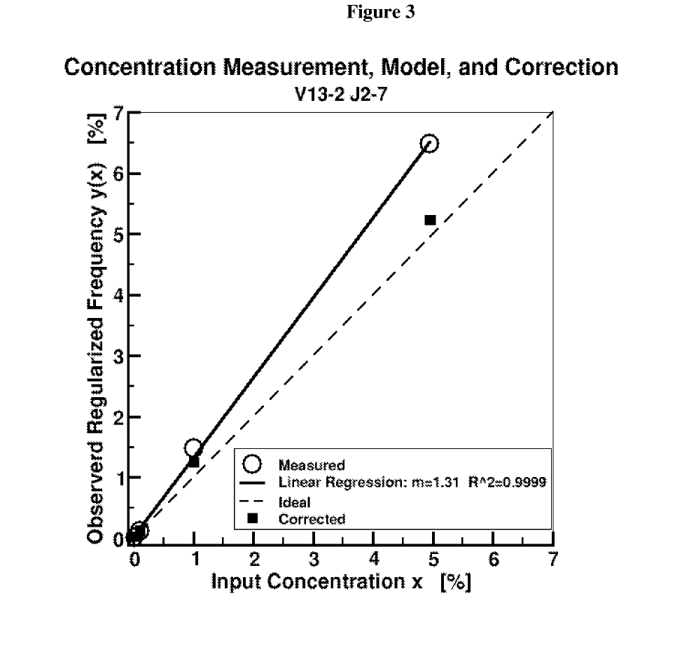

FIG. 3 shows empirical measurements made for one V-J pair at four different concentrations (circles). A linear model is fit to the measurements (solid line). If the measurements are unbiased, the assumption is that the data follow y(x)=x (dashed line). Such an assumption would lead to false conclusions from the empirical data. The linear model is used to correct bias informatically, reconciling the biased measurements (circles) with the unbiased case (dashed line) as demonstrated by the corrected data (solid squares).

Many different mathematical models can be built, depending on the empirical behavior of the amplification reaction. Linear or nonlinear model may appropriately fit certain data sets, and various statistical methods could be used to fit the data.

One of ordinary skill could readily obtain the sequences for the PCR probes and primers from databases such as RefSeq (http://www.ncbi.nlm.nih.gov/gene/), the international ImMunoGeneTics information System® (http://www.imgt.org/), EMBL Nucleotide Sequence Database VBASE2 (http://www.vbase2.org/), or MRC Centre for Protein Engineering V BASE (http://vbase.mrc-cpe.cam.ac.uk/).

### 6.4. Computer Implemented Methods

The computer-implemented method or system may be configured in either hardware, software, or both based on the types of applications needed and the hardware available. Hardware examples of implementation include hardware implemented ASIC (“Application Specific Integrated Circuit”), SOC (“System on a Chip”), RISC (“Reduced Instruction Set Computing”) processor, general processor, DSP (“Digital Signal Processor”), etc.

The various implementations of the subject matter disclosed herein may be implemented in hardware, software, or both. In the present context, software comprises an ordered listing of executable instructions for implementing logical functions, and may selectively be embodied in any computer-readable medium for use by or in connection with an instruction execution system, apparatus, or device, such as a computer-based system, processor-containing system, or other system that may selectively fetch the instructions from the instruction execution system, apparatus, or device and execute the instructions. In the context of this document, a “computer-readable medium” is any means that may contain, store, communicate, propagate, or transport the program for use by or in connection with the instruction execution system, apparatus, or device. The computer-readable medium may selectively be, for example but not limited to, an electronic, magnetic, optical, electromagnetic, infrared, or semiconductor system, apparatus, device, or propagation medium. More specific (yet a non-exhaustive list of) examples of the computer-readable medium would include the following: an electrical connection (electronic) having one or more wires, a portable computer diskette (magnetic), a RAM (electronic), a read-only memory “ROM” (electronic), an erasable programmable read-only memory (EPROM or Flash memory) (electronic), an optical fiber (optical), and a portable compact disc read-only memory “CDROM” (optical).

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

It also is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A method for preparing a series of mathematical functions for correction of bias in amplification of a plurality of immune related sequences which comprises:
a. amplifying a first mixture comprising at least two different immune related sequences at known concentrations;
b. amplifying a second mixture comprising the immune related sequences of step (a) wherein the sequences are present at different concentrations than the first mixture;
c. measuring sequence counts for the first and second amplified mixtures of immune related sequences;
d. generating a plurality of mathematical functions for correction of bias that model relationships between concentrations and measured sequence counts;
e. assembling the mathematical functions of step (d) to generate the series of mathematical functions useful to correct amplification bias.

2. The method of claim 1, wherein immune related sequences are subcloned into a circular vector.

3. The method of claim 1, wherein multiplexed polymerase chain reaction is used to amplify the immune related sequences.

4. The method of claim 1, wherein the immune related sequences are immunoglobulin IgH or immunoglobulin IgL sequences.

5. The method of claim 1, wherein the immune related sequences are T cell receptor sequences.

6. The method of claim 1, wherein the immune related sequences are joining (J) gene sequences.

7. The method of claim 1, wherein the immune related sequences are variable (V) gene sequences.

8. The method of claim 1, wherein greater than forty immune related sequences are selected from possible combinations of joining (J) and variable (V) gene sequences.

9. The method of claim 1, wherein more than six different immune related sequences are used in the first mixture and the concentration differences for at least one immune related sequence is greater than three orders of magnitude in the second mixture.

10. The method of claim 1, where the mathematical function is a linear or nonlinear equation.

11. A method for correction of bias in amplification of in an immune repertoire sample, comprising:
a. amplifying of an immune repertoire sample;
b. obtaining at least 1,000 sequences from the amplified immune repertoire sample; and
c. correcting levels generated from amplification at least one sequence in the immune repertoire sample using at least one mathematical function from the series of mathematical functions generated in claim 1.

12. The method of claim 11, wherein massively parallel sequencing is used to generate sequences from the immune repertoire sample.

13. The method of claim 11, wherein at least 10,000 sequences are obtained from the immune repertoire sample.

14. The method of claim 11, wherein at least 100,000 sequences are obtained from the immune repertoire sample.

15. A computer-implemented method for correcting bias in a plurality of immune related sequences which comprises:
a. obtaining a plurality of measurements of levels of amplified immune related sequences from an unknown sample; and
b. using an assembled series of mathematical functions and the measurements from step (a) to correct amplification bias in amplified immune related sequences from the unknown sample.

16. The computer-implemented method for correcting bias of claim 15, wherein step (a) and step (b) are carried out automatically.

17. A system for correcting bias in amplified immune related sequences which comprises:
a. an assembler module comprising an assembled series of mathematical functions useful to correct amplification bias in a plurality of immune related sequences; and
b. a calculating module that corrects bias in amplification of immune related sequences using the assembled series of mathematical functions and measurements of levels of amplified immune related sequences from an unknown sample.
